(19) United States
(12) Patent Application Publication
(10) Pub. No.: US 2021/0109068 A1
(43) Pub. Date: Apr. 15, 2021

(54) FINGERPRINT DETECTION METHOD FOR ANTIDEPRESSANT DRUG

(71) Applicants: Chenland Nutritionals Inc., Pomona, CA (US); Qingdao Chenland Pharmaceutical Technology Development Co., Ltd., Qingdao (CN)

(72) Inventors: Shengcan Zou, Qingdao (CN); Jiancheng Zong, Qingdao (CN); Wenyu Li, Qingdao (CN); Lei Zong, Qingdao (CN); Zengliang Zhang, Qingdao (CN); Shanglong Wang, Qingdao (CN)

(73) Assignees: Chenland Nutritionals Inc.; Qingdao Chenland Pharmaceutical Technology Development Co., Ltd.

(21) Appl. No.: 17/094,840

(22) Filed: Nov. 11, 2020

(51) Int. Cl. G01N 30/86; B01J 20/286; B01J 20/10; A61K 36/185; A61K 36/65; A61K 36/744; A61K 36/48; G01N 30/06; G01N 30/60

(52) U.S. Cl. CPC G01N 30/8631; B01J 20/286; B01J 20/103; A61K 36/185; A61K 36/65; G01N 2030/027; A61K 36/48; G01N 30/06; G01N 30/60; B01J 2220/54; B01J 2220/58; A61K 36/744

(57) ABSTRACT

The present invention discloses a fingerprint detection method for an antidepressant drug, which adopts liquid chromatography to characterize pharmacological active ingredients of the antidepressant drug composed of Hypericum perforatum L., Paeonia lactiflora Pall, Gardenia jasminoides Ellis, Albizia julibrissin Durazz, and Moutan Cortex, provides an effective detection method, and realizes the quality control of traditional Chinese medicine compositions. The fingerprint detection method provided by the present invention has the advantages of high repeatability and high stability.

### TECHNICAL FIELD

The present invention relates to the field of analysis of traditional Chinese medicines, and more particularly relates to a fingerprint detection method for an antidepressant drug.

### BACKGROUND

The traditional Chinese medicine fingerprint refers to an atlas obtained by means of modern information collection technology and quality analysis means after appropriate treatment of some traditional Chinese medicines or Chinese patent medicines and capable of displaying images, figures, spectrograms and data of chemical features of the traditional Chinese medicines or the Chinese patent medicines. As a quality control mode, the traditional Chinese medicine fingerprint is gradually valued by people. The most basic attributes in the chromatographic fingerprints are integrity and ambiguity. It is a comprehensive and quantizable identification means, is based on system research of chemical components of the traditional Chinese medicine and is mainly used for evaluating the authenticity, the superiority and the stability of the quality of semi-finished products of the traditional Chinese medicines and the traditional Chinese medicine formulations. The fingerprint atlas should have fingerprint characteristics, namely: (1) feature and specificity, which means that the designated fingerprint atlas is unique to this traditional Chinese medicine and is different from other traditional Chinese medicines, such as differences in species, production place, or harvest season, etc., and the chemical information presented by the atlas is highly selective; (2) stability and reproducibility: the traditional Chinese medicine fingerprint should represent the commonality summarized from multiple batches of a certain traditional Chinese medicine, under the prescribed methods and conditions, the common peaks or characteristic peaks in the atlas should be relatively stable; (3) validity and integrity: the specified fingerprint atlas should be able to reproduce fingerprint characteristics (such as number, size, position, etc. of common peaks) under specified conditions, and the error thereof should be within the allowable range.

Depression mood, as a common mood disorder disease, is an affective disorder disease with low mood as the main symptom and corresponding thinking and behavior changes, has the clinical manifestations including low mood, retardation of thinking, decreased appetite, sleep disorder, etc., which seriously endanger people's physical and mental health. Nowadays, as the pace of life is accelerating, and employment competition is intensifying, the number of people experiencing depression is increasing year by year. Severe depression mood can develop into depression and even suicide.

The fingerprint method has certain limitations to drug detection, for example, the quality of Chinese medicinal materials varies greatly, different batches and production places also have significant influence on the detection of fingerprints; the detection of fingerprints is greatly affected by environments and instruments, the established standards cannot meet the requirements of various time periods or different laboratories; in addition, the standardization from extraction and separation to analysis and detection of medicinal materials is not enough, which also limits the detection of fingerprints.

Therefore, with respect to the above problems, the problem to be urgently solved by those skilled in the art is to provide a fingerprint detection method for an antidepressant drug.

### SUMMARY

In view of this, the present invention provides a fingerprint detection method for an antidepressant drug. The method uses high performance liquid chromatography to effectively characterize the pharmacological active ingredients of Hypericum perforatum L., Paeonia lactiflora Pall, Gardenia jasminoides Ellis, Albizia julibrissin Durazz, and Moutan Cortex, and can effectively realize the quality control of a detected traditional Chinese medicine composition. The fingerprint detection method has the advantages of high repeatability and high stability.

To achieve the above purpose, the present invention adopts the following technical solution:

A fingerprint detection method for an antidepressant drug, wherein liquid chromatography is adopted with chromatographic conditions are as follows:

a chromatographic column adopts octadecylsilane chemically bonded silica as a filler;

mobile phase A is acetonitrile, mobile phase B is phosphoric acid aqueous solution, and gradient elution is adopted;

detection wavelength is 230-300 nm;

flow rate is 1 ml/min;

sample injection volume is 10 μL;

the number of theoretical plates is based on peaks of standard hyperoside, ≥3000;

preparation of a standard solution: taking hyperoside, accurately weighing same, and adding 50% methanol aqueous solution to prepare a solution with a concentration of 1 mg/ml;

gradient elution conditions are as follows:

| Time, min Mobile Phase A %, | Acetonitrile Mobile Phase B %, | 0.1% Phosphoric Acid |
|---|---|---|
| 0 | 5 | 95 |
| 5 | 5 | 95 |
| 15 | 14 | 86 |
| 25 | 14 | 86 |
| 30 | 15 | 85 |
| 35 | 15 | 85 |
| 40 | 20 | 80 |
| 75 | 20 | 80 |
| 80 | 25 | 75 |
| 90 | 25 | 75 |
| 95 | 37 | 63 |
| 115 | 37 | 63 |
| 135 | 90 | 10. |

Preferably, the antidepressant drug is composed of the following parts by weight of raw materials: 3 parts of Hypericum perforatum L., 12 parts of Paeonia lactiflora Pall, 6 parts of Gardenia jasminoides Ellis, 10 parts of Moutan Cortex and 10 parts of Albizia julibrissin Durazz.

Preferably, the specific preparation steps of the antidepressant drug are as follows:

S1: weighing Hypericum perforatum L., Paeonia lactiflora Pall, Gardenia jasminoides Ellis, Albizia julibrissin Durazz, and Moutan Cortex in proportion to prepare materials;

S2: adding 10 times the amount of 60% ethanol to the Hypericum perforatum L., conducting heating reflux extraction twice for 1.5 h each time; merging the extracting solutions, concentrating, and drying under reduced pressure; adding 10 times the amount of 70% ethanol to the Paeonia lactiflora Pall, conducting heating reflux extraction twice for 1.5 h each time, merging the extracting solutions, concentrating, and drying under reduced pressure; adding 10 times the amount of 80% ethanol to the Gardenia jasminoides Ellis, conducting heating reflux extraction twice for 1.5 h each time, merging the extracting solutions, concentrating, and drying under reduced pressure; adding 10 times the amount of pure water to the Albizia julibrissin Durazz., conducting heating reflux extraction twice for 1.5 h each time, merging the extracting solutions, concentrating, and drying under reduced pressure; and adding 10 times the amount of pure water to the Moutan Cortex, and obtaining an extract by steam distillation.

S3: grinding and mixing the obtained extracts A-E at a weight ratio of 15:14:7:5:0.6 uniformly, and sieving the mixed extract with an 80-mesh pharmacopoeia sieve, obtaining an antidepressant compound preparation.

Preferably, the fingerprint of the antidepressant drug is:

the fingerprint comprises 15 common peaks: the relative retention times of all peaks are: (1) 9.153, (2) 11.695, (3) 15.111, (4) 16.559, (5) 19.563, (6) 20.383, (7) 32.511, (8) 44.247, (9S) 44.856, (10) 45.764, (11) 49.397, (12) 51.643, (13) 53.324, (14) 88.658, (15) 96.463.

Preferably, the common peaks of the fingerprint include:

Peak 1: having a retention time of 9.153, an RSD of 0.08%, and a peak area of 1336.402, which accounts for 4.432% of the total peak area;

Peak 2: having a retention time of 11.695, an RSD of 0.04%, and a peak area of 521.328, which accounts for 1.729% of the total peak area;

Peak 3: having a retention time of 15.111, an RSD of 0.04%, and a peak area of 305.260, which accounts for 1.012% of the total peak area;

Peak 4: having a retention time of 16.559, an RSD of 0.04%, and a peak area of 437.930, which accounts for 1.452% of the total peak area;

Peak 5: having a retention time of 19.563, an RSD of 0.06%, and a peak area of 406.334, which accounts for 1.348% of the total peak area;

Peak 6: having a retention time of 20.383, an RSD of 0.04%, and a peak area of 1151.954, which accounts for 3.820% of the total peak area;

Peak 7: having a retention time of 32.511, an RSD of 0.04%, and a peak area of 820.504, which accounts for 2.721% of the total peak area;

Peak 8: having a retention time of 44.247, an RSD of 0.09%, and a peak area of 1617.883, which accounts for 5.365% of the total peak area;

Peak 9S: having a retention time of 44.856, an RSD of 0.07%, and a peak area of 1299.504, which accounts for 4.309% of the total peak area;

Peak 10: having a retention time of 45.764, an RSD of 0.09%, and a peak area of 1299.504, which accounts for 4.433% of the total peak area;

Peak 11: having a retention time of 49.397, an RSD of 0.09%, and a peak area of 2051.489, which accounts for 6.803% of the total peak area;

Peak 12: having a retention time of 51.643, an RSD of 0.05%, and a peak area of 563.488, which accounts for 1.869% of the total peak area;

Peak 13: having a retention time of 53.324, an RSD of 0.03%, and a peak area of 895.859, which accounts for 2.971% of the total peak area;

Peak 14: having a retention time of 88.658, an RSD of 0.02%, and a peak area of 646.792, which accounts for 2.145% of the total peak area; and

Peak 15: having a retention time of 96.463, an RSD of 0.04%, and a peak area of 9089.165, which accounts for 30.142% of the total peak area.

Preferably, in the method, the pH of the mobile phase A is 2.0-7.0.

Preferably, in the method, the pH of the mobile phase A is 2.5.

Preferably, for a reference solution used for determination by liquid chromatography in the method, a preparation method thereof includes: taking hyperoside and putting same into a volumetric flask, adding methanol aqueous solution, conducting ultrasonic treatment for dissolution, making constant volume, filtering, thus obtaining a filtrate as the reference solution.

Preferably, for a test solution used for determination by liquid chromatography in the method, a preparation method thereof includes: taking dry powder of the antidepressant drug and putting same into a volumetric flask, adding methanol aqueous solution, conducting ultrasonic treatment for dissolution, making constant volume, filtering, thus obtaining a filtrate as the test solution.

Preferably, the methanol aqueous solution is 40%-60% methanol solution.

It can be known from the above technical solution that compared with the prior art, the present invention has the following beneficial effects:

1. The present invention creates a fingerprint detection method for a traditional Chinese medicine composition which uses high performance liquid chromatography and a fingerprint thereof, effectively characterizes the pharmacological active ingredients of Hypericum perforatum L., Paeonia lactiflora Pall, Gardenia jasminoides Ellis, Albizia julibrissin Durazz., provides an effective detection method, and realizes the quality control of some Traditional Chinese medicine compositions.

2. The fingerprint detection method provided by the present invention has high repeatability and stability.

3. By means of the fingerprint detection method, different batches of medicinal materials from different production places can be effectively monitored, providing a reference for the incoming inspection of medicinal materials.

4. It is proved by animal efficacy experiments that the traditional Chinese medicine composition of the present invention is helpful to relieve depression mood and has an antidepressant effect, the present invention develops a quality control method for an antidepressant traditional Chinese medicine composition on the basis of drug efficacy, not only provides a basis for clearly elucidating the relationship between the fingerprint characteristics and drug efficacy, but also can effectively control the quality of the active ingredients of the traditional Chinese medicine composition, providing a theoretical basis for the expanded production and clinical rational use of the traditional Chinese medicine composition.

### DESCRIPTION OF DRAWINGS

To more clearly describe the technical solution in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 shows the fingerprint of the Hypericum perforatum L. extract in embodiment 1 of the present invention;

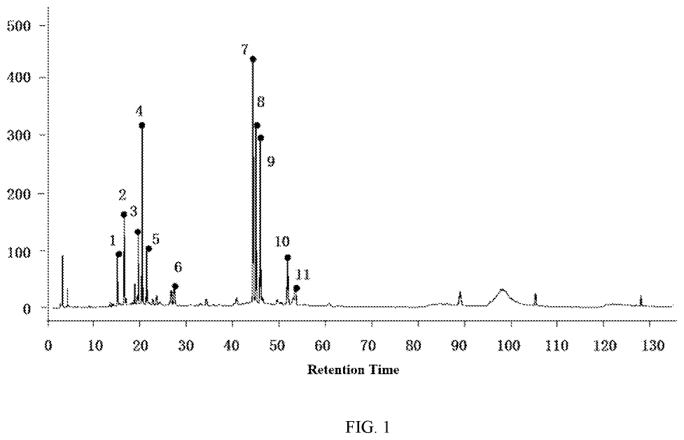

FIG. 2 shows the fingerprint of the Paeonia lactiflora Pall extract in embodiment 2 of the present invention;

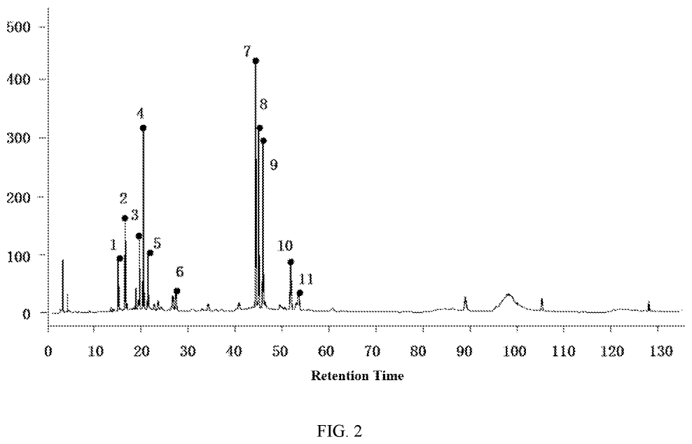

FIG. 3 shows the fingerprint of the Gardenia jasminoides Ellis extract in embodiment 3 of the present invention;

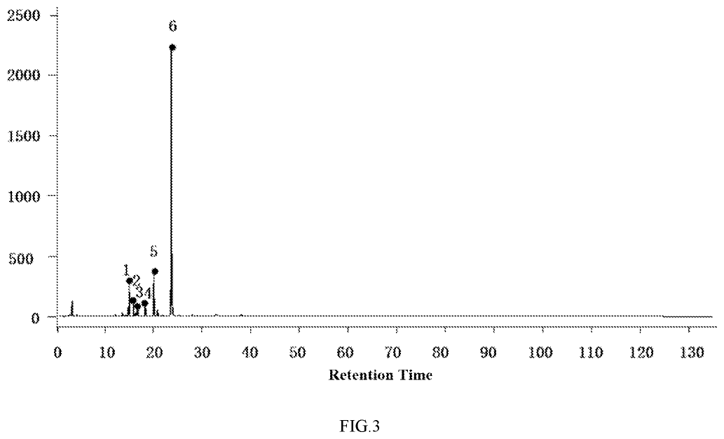

FIG. 4 shows the fingerprint of the Moutan Cortex extract in embodiment 4 of the present invention;

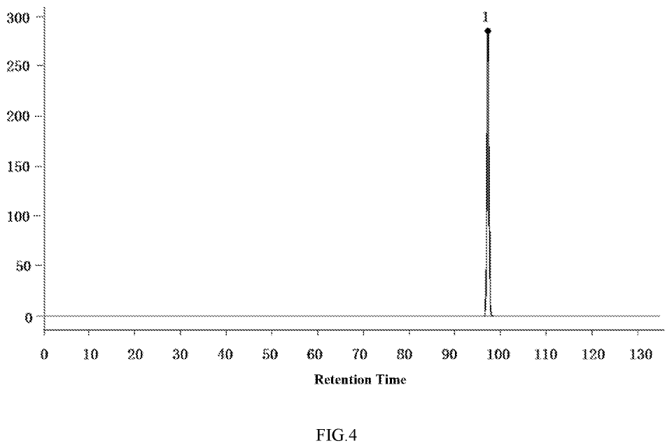

FIG. 5 shows the fingerprint of the Albizia julibrissin Durazz. extract in embodiment 5 of the present invention; and

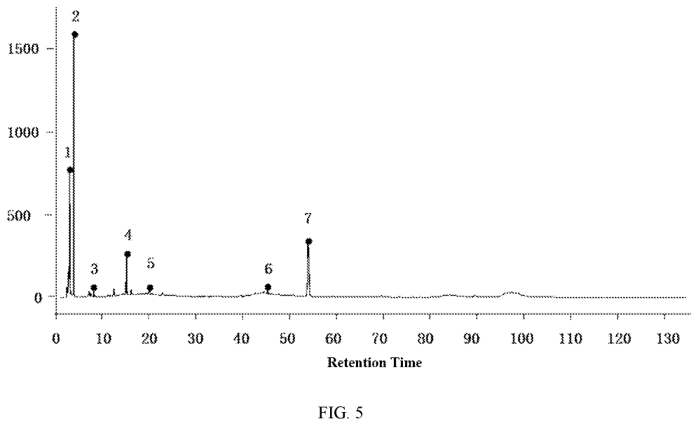

FIG. 6 shows the fingerprint of the traditional Chinese medicine composition in embodiment 6 of the present invention.

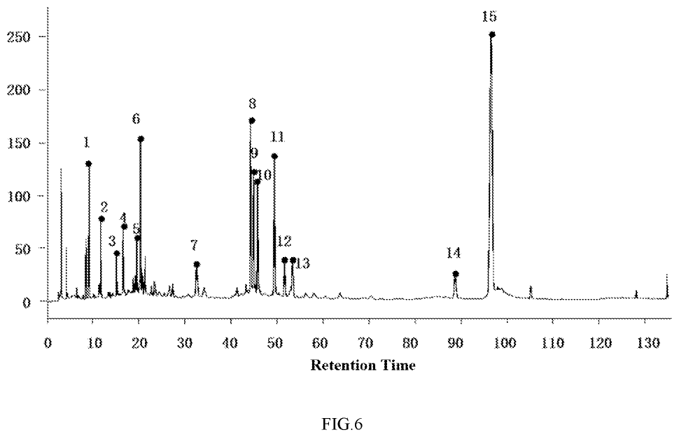

### DETAILED DESCRIPTION

The technical solution in embodiments of the present invention will be clearly and fully described below. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

### Embodiment 1

Embodiment 1 of the present invention discloses fingerprint determination for Hypericum perforatum L. The adopted technical solution is as follows:

1. Preparation Method

Adding 10 times the amount of 60% ethanol to the Hypericum perforatum L., conducting heating reflux extraction twice for 1.5 h each time, merging the extracting solutions, concentrating, and drying under reduced pressure; weighing 0.71 g, putting in a 25 ml volumetric flask, adding 50% methanol aqueous solution, conducting ultrasonic treatment for 20 m for dissolution, adding 50% methanol water to the mark, filtering, obtaining a sample solution of the Hypericum perforatum L. extract.

2. Main Instruments

Chromatographic system: Agilent Technologies 1260 Infinity II (G7111B 1260 Quat Pump, G7129A 1260 Vialsampler, G7116A 1260 MCT, G7117B 1290 DAD);

Ultrasonic cleaner: KQ-600DE, Kun Shan Ultrasonic Instruments Co., Ltd.;

Rotary evaporator: RE-52A, Shanghai Yarong Biochemistry Instrument Company;

Circulating water vacuum pump: SHZ-D(III), Gongyi Yuhua Instrument Co., Ltd.

3. Reagents

Methanol: 20180606, purity≥99.9%, Sinopharm Chemical Reagent Co., Ltd.;

Acetonitrile: 20180731, purity≥99.9%, Sinopharm Chemical Reagent Co., Ltd.;

Phosphoric acid: purity≥85%, Tianjin Kermel Chemical Reagent Co., Ltd.;

Purified water: 20180927, A.S. Watson Group.

4. Chromatographic Conditions:

A chromatographic column adopts octadecylsilane chemically bonded silica as a filler; gradient elution is adopted; mobile phase A is acetonitrile; mobile phase B is phosphoric acid water; detection wavelength is 300 nm; flow rate is 1 ml/min; column temperature is 30° C.; and sample injection volume is 10 μL; and gradient elution procedures are:

| Time, min Mobile Phase A %, | Acetonitrile Mobile Phase B %, | 0.1% Phosphoric Acid |
|---|---|---|
| 0 | 5 | 95 |
| 5 | 5 | 95 |
| 15 | 14 | 86 |
| 25 | 14 | 86 |
| 30 | 15 | 85 |
| 35 | 15 | 85 |
| 40 | 20 | 80 |
| 75 | 20 | 80 |
| 80 | 25 | 75 |
| 90 | 25 | 75 |
| 95 | 37 | 63 |
| 115 | 37 | 63 |
| 135 | 90 | 10. |

5. Determination

Sucking the sample solution of the Hypericum perforatum L. extract and injecting same in a liquid phase chromatograph, determining according to high performance liquid chromatography, and obtaining a fingerprint of the Hypericum perforatum L. extract, wherein the fingerprint peaks include:

Peak 1: having a retention time RT of 14.972, an RSD of 0.8%, and a peak area of 755.783, which accounts for 2.603% of the total peak area;

Peak 2: having a retention time RT of 16.424, an RSD of 0.4%, and a peak area of 1195.265, which accounts for 4.116% of the total peak area;

Peak 3: having a retention time RT of 19.465, an RSD of 0.3%, and a peak area of 1032.728, which accounts for 3.556% of the total peak area;

Peak 4: having a retention time RT of 20.287, an RSD of 0.4%, and a peak area of 2684.446, which accounts for 9.244% of the total peak area;

Peak 5: having a retention time RT of 21.278, an RSD of 0.5%, and a peak area of 968.582, which accounts for 3.335% of the total peak area;

Peak 6: having a retention time RT of 26.564, an RSD of 0.2%, and a peak area of 523.280, which accounts for 1.802% of the total peak area;

Peak 7: having a retention time RT of 44.227, an RSD of 0.3%, and a peak area of 4200.473, which accounts for 14.464% of the total peak area;

Peak 8: having a retention time RT of 44.847, an RSD of 0.3%, and a peak area of 3439.290, which accounts for 11.843% of the total peak area;

Peak 9: having a retention time RT of 45.769, an RSD of 0.5%, and a peak area of 3686.598, which accounts for 12.695% of the total peak area;

Peak 10: having a retention time RT of 51.697, an RSD of 0.4%, and a peak area of 1622.576, which accounts for 5.587% of the total peak area; and

Peak 11: having a retention time RT of 53.377, an RSD of 0.03%, and a peak area of 737.842, which accounts for 2.541% of the total peak area.

### Embodiment 2

Embodiment 2 of the present invention discloses fingerprint determination for a Paeonia lactiflora Pall extract. The adopted technical solution is as follows:

1. Preparation Method

Adding 10 times the amount of 70% ethanol to the Paeonia lactiflora Pall, conducting heating reflux extraction twice for 1.5 h each time, merging the extracting solutions, concentrating, and drying under reduced pressure; weighing 0.71 g, putting in a 25 ml volumetric flask, adding 50% methanol aqueous solution, conducting ultrasonic treatment for 20 m for dissolution, adding 50% methanol water to the mark, filtering, obtaining a sample solution of the Paeonia lactiflora Pall extract.

2. Main Instruments

Chromatographic system: Agilent Technologies 1260 Infinity II (G7111B 1260 Quat Pump, G7129A 1260 Vialsampler, G7116A 1260 MCT, G7117B 1290 DAD);

Ultrasonic cleaner: KQ-600DE, Kun Shan Ultrasonic Instruments Co., Ltd.;

Rotary evaporator: RE-52A, Shanghai Yarong Biochemistry Instrument Company

Circulating water vacuum pump: SHZ-D(III), Gongyi Yuhua Instrument Co., Ltd.

3. Reagents

Methanol: 20180606, purity≥99.9%, Sinopharm Chemical Reagent Co., Ltd.;

Acetonitrile: 20180731, purity≥99.9%, Sinopharm Chemical Reagent Co., Ltd.;

Phosphoric acid: purity≥85%, Tianjin Kermel Chemical Reagent Co., Ltd.;

Purified water: 20180927, A.S. Watson Group.

4. Chromatographic conditions:

A chromatographic column adopts octadecylsilane chemically bonded silica as a filler; gradient elution is adopted; mobile phase A is acetonitrile; mobile phase B is phosphoric acid water; detection wavelength is 230 nm; flow rate is 1 ml/min; column temperature is 30° C.; sample injection volume is 10 μL; gradient elution procedures are:

| Time, min Mobile Phase A %, | Acetonitrile Mobile Phase B %, | 0.1% Phosphoric Acid |
|---|---|---|
| 0 | 5 | 95 |
| 5 | 5 | 95 |
| 15 | 14 | 86 |
| 25 | 14 | 86 |
| 30 | 15 | 85 |
| 35 | 15 | 85 |
| 40 | 20 | 80 |
| 75 | 20 | 80 |
| 80 | 25 | 75 |
| 90 | 25 | 75 |
| 95 | 37 | 63 |
| 115 | 37 | 63 |
| 135 | 90 | 10. |

5. Determination

Sucking the sample solution of the Paeonia lactiflora Pall extract and injecting same in a liquid phase chromatograph, determining according to high performance liquid chromatography, and obtaining a fingerprint of the Paeonia lactiflora Pall extract, wherein the fingerprint peaks include:

Peak 1: having a retention time RT of 9.374, an RSD of 0.5%, and a peak area of 9410.198, which accounts for 5.785% of the total peak area;

Peak 2: having a retention time RT of 11.937, an RSD of 0.9%, and a peak area of 2016.463, which accounts for 1.240% of the total peak area;

Peak 3: having a retention time RT of 27.011, an RSD of 0.5%, and a peak area of 22562.261, which accounts for 13.871% of the total peak area;

Peak 4: having a retention time RT of 30.912, an RSD of 0.6%, and a peak area of 68138.645, which accounts for 41.892% of the total peak area;

Peak 5: having a retention time RT of 44.947, an RSD of 0.7%, and a peak area of 18202.291, which accounts for 11.191% of the total peak area;

Peak 6: having a retention time RT of 50.365, an RSD of 0.6%, and a peak area of 4852.267, which accounts for 2.983% of the total peak area;

Peak 7: having a retention time RT of 56.724, an RSD of 0.5%, and a peak area of 2196.713, which accounts for 1.351% of the total peak area; and

Peak 8: having a retention time RT of 99.365, an RSD of 0.2%, and a peak area of 1948.910, which accounts for 1.198% of the total peak area.

### Embodiment 3

Embodiment 3 of the present invention discloses fingerprint determination for Gardenia jasminoides Ellis extract. The adopted technical solution is as follows:

1. Preparation Method

Adding 10 times the amount of 80% ethanol to the Gardenia jasminoides Ellis, conducting heating reflux extraction twice for 1.5 h each time, merging the extracting solutions, concentrating, and drying under reduced pressure; weighing 0.71 g, putting in a 25 ml volumetric flask, adding 50% methanol aqueous solution, conducting ultrasonic treatment for 20 m for dissolution, adding 50% methanol water to the mark, filtering, obtaining a sample solution of the Gardenia jasminoides Ellis extract.

2. Main Instruments

Chromatographic system: Agilent Technologies 1260 Infinity II (G7111B 1260 Quat Pump, G7129A 1260 Vialsampler, G7116A 1260 MCT, G7117B 1290 DAD);

Ultrasonic cleaner: KQ-600DE, Kun Shan Ultrasonic Instruments Co., Ltd.;

Rotary evaporator: RE-52A, Shanghai Yarong Biochemistry Instrument Company

Circulating water vacuum pump: SHZ-D(III), Gongyi Yuhua Instrument Co., Ltd.

3. Reagents

Methanol: 20180606, purity≥99.9%, Sinopharm Chemical Reagent Co., Ltd.;

Acetonitrile: 20180731, purity≥99.9%, Sinopharm Chemical Reagent Co., Ltd.;

Phosphoric acid: purity≥85%, Tianjin Kermel Chemical Reagent Co., Ltd.;

Purified water: 20180927, A.S. Watson Group.

4. Chromatographic Conditions:

A chromatographic column adopts octadecylsilane chemically bonded silica as a filler; gradient elution is adopted; mobile phase A is acetonitrile; mobile phase B is phosphoric acid water; detection wavelength is 300 nm; flow rate is 1 ml/min; column temperature is 30° C.; sample injection volume is 10 μL; gradient elution procedures are:

| Time, min Mobile Phase A %, | Acetonitrile Mobile Phase B %, | 0.1% Phosphoric Acid |
|---|---|---|
| 0 | 5 | 95 |
| 5 | 5 | 95 |
| 15 | 14 | 86 |
| 25 | 14 | 86 |
| 30 | 15 | 85 |
| 35 | 15 | 85 |
| 40 | 20 | 80 |
| 75 | 20 | 80 |
| 80 | 25 | 75 |
| 90 | 25 | 75 |
| 95 | 37 | 63 |
| 115 | 37 | 63 |
| 135 | 90 | 10. |

5. Determination

Sucking the sample solution of the Gardenia jasminoides Ellis extract and injecting same in a liquid phase chromatograph, determining according to high performance liquid chromatography, and obtaining a fingerprint of the Gardenia jasminoides Ellis extract, wherein the fingerprint peaks include:

Peak 1: having a retention time RT of 14.912, an RSD of 0.4%, and a peak area of 1933.598, which accounts for 4.781% of the total peak area;

Peak 2: having a retention time RT of 15.761, an RSD of 0.4%, and a peak area of 819.484, which accounts for 2.026% of the total peak area;

Peak 3: having a retention time RT of 16.615, an RSD of 0.2%, and a peak area of 608.844, which accounts for 1.505% of the total peak area;

Peak 4: having a retention time RT of 18.178, an RSD of 0.5%, and a peak area of 907.286, which accounts for 2.243% of the total peak area;

Peak 5: having a retention time RT of 19.971, an RSD of 0.8%, and a peak area of 2888.059, which accounts for 7.141% of the total peak area; and

Peak 6: having a retention time RT of 32.594, an RSD of 0.8%, and a peak area of 27584.143, which accounts for 68.205% of the total peak area.

### Embodiment 4

Embodiment 4 of the present invention discloses fingerprint determination for a Moutan Cortex extract. The adopted technical solution is as follows:

1. Preparation Method

Adding 10 times the amount of pure water to the Moutan Cortex, and obtaining an extract by steam distillation.

2. Main Instruments

Chromatographic system: Agilent Technologies 1260 Infinity II (G7111B 1260 Quat Pump, G7129A 1260 Vialsampler, G7116A 1260 MCT, G7117B 1290 DAD);

Ultrasonic cleaner: KQ-600DE, Kun Shan Ultrasonic Instruments Co., Ltd.;

Rotary evaporator: RE-52A, Shanghai Yarong Biochemistry Instrument Company

Circulating water vacuum pump: SHZ-D(III), Gongyi Yuhua Instrument Co., Ltd.

3. Reagents

Methanol: 20180606, purity≥99.9%, Sinopharm Chemical Reagent Co., Ltd.;

Acetonitrile: 20180731, purity≥99.9%, Sinopharm Chemical Reagent Co., Ltd.;

Phosphoric acid: purity≥85%, Tianjin Kermel Chemical Reagent Co., Ltd.;

Purified water: 20180927, A.S. Watson Group.

4. Chromatographic Conditions:

A chromatographic column adopts octadecylsilane chemically bonded silica as a filler; gradient elution is adopted; mobile phase A is acetonitrile; mobile phase B is phosphoric acid water; detection wavelength is 274 nm; flow rate is 1 ml/min; column temperature is 30° C.; sample injection volume is 10 μL; gradient elution procedures are:

| Time, min Mobile Phase A %, | Acetonitrile Mobile Phase B %, | 0.1% Phosphoric Acid |
|---|---|---|
| 0 | 5 | 95 |
| 5 | 5 | 95 |
| 15 | 14 | 86 |
| 25 | 14 | 86 |
| 30 | 15 | 85 |
| 35 | 15 | 85 |
| 40 | 20 | 80 |
| 75 | 20 | 80 |
| 80 | 25 | 75 |
| 90 | 25 | 75 |
| 95 | 37 | 63 |
| 115 | 37 | 63 |
| 135 | 90 | 10. |

5. Determination

Sucking the sample solution of the Moutan Cortex extract and injecting same in a liquid phase chromatograph, determining according to high performance liquid chromatography, and obtaining a fingerprint of the Moutan Cortex extract, wherein the fingerprint peaks include:

Peak 1: having a retention time RT of 97.110, an RSD of 0.09%, and a peak area of 3076.943, which accounts for 99.650% of the total peak area.

### Embodiment 5

Embodiment 5 of the present invention discloses fingerprint determination for Albizia julibrissin Durazz. The adopted technical solution is as follows:

1. Preparation Method

Adding 10 times the amount of pure water to the Albizia julibrissin Durazz., conducting heating reflux extraction twice for 1.5 h each time, merging the extracting solutions, concentrating, and drying under reduced pressure; weighing 0.71 g, putting in a 25 ml volumetric flask, adding 50% methanol aqueous solution, conducting ultrasonic treatment for 20 m for dissolution, adding 50% methanol water to the mark, filtering, obtaining a sample solution of the Albizia julibrissin Durazz. extract.

2. Main Instruments

Chromatographic system: Agilent Technologies 1260 Infinity II (G7111B 1260 Quat Pump, G7129A 1260 Vialsampler, G7116A 1260 MCT, G7117B 1290 DAD);

Ultrasonic cleaner: KQ-600DE, Kun Shan Ultrasonic Instruments Co., Ltd.;

Rotary evaporator: RE-52A, Shanghai Yarong Biochemistry Instrument Company

Circulating water vacuum pump: SHZ-D(III), Gongyi Yuhua Instrument Co., Ltd.

3. Reagents

Methanol: 20180606, purity≥99.9%, Sinopharm Chemical Reagent Co., Ltd.;

Acetonitrile: 20180731, purity≥99.9%, Sinopharm Chemical Reagent Co., Ltd.;

Phosphoric acid: purity≥85%, Tianjin Kermel Chemical Reagent Co., Ltd.;

Purified water: 20180927, A.S. Watson Group.

4. Chromatographic Conditions:

A chromatographic column adopts octadecylsilane chemically bonded silica as a filler; gradient elution is adopted; mobile phase A is acetonitrile; mobile phase B is phosphoric acid water; detection wavelength is 256 nm; flow rate is 1 ml/min; column temperature is 30° C.; sample injection volume is 10 μL; gradient elution procedures are:

| Time, min Mobile Phase A %, | Acetonitrile Mobile Phase B %, | 0.1% Phosphoric Acid |
|---|---|---|
| 0 | 5 | 95 |
| 5 | 5 | 95 |
| 15 | 14 | 86 |
| 25 | 14 | 86 |
| 30 | 15 | 85 |
| 35 | 15 | 85 |
| 40 | 20 | 80 |
| 75 | 20 | 80 |
| 80 | 25 | 75 |
| 90 | 25 | 75 |
| 95 | 37 | 63 |
| 115 | 37 | 63 |
| 135 | 90 | 10. |

5. Determination

Sucking the sample solution of the Albizia julibrissin Durazz. extract and injecting same in a liquid phase chromatograph, determining according to high performance liquid chromatography, and obtaining a fingerprint of the Albizia julibrissin Durazz. extract, wherein the fingerprint peaks include:

Peak 1: having a retention time RT of 3.133, an RSD of 0.5%, and a peak area of 3036.946, which accounts for 10.294% of the total peak area;

Peak 2: having a retention time RT of 3.956, an RSD of 0.3%, and a peak area of 5068.172, which accounts for 17.179% of the total peak area;

Peak 3: having a retention time RT of 7.329, an RSD of 0.2%, and a peak area of 536.413, which accounts for 1.818% of the total peak area;

Peak 4: having a retention time RT of 15.243, an RSD of 0.2%, and a peak area of 2024.062, which accounts for 6.861% of the total peak area;

Peak 5: having a retention time RT of 20.146, an RSD of 0.5%, and a peak area of 396.857, which accounts for 1.345% of the total peak area;

Peak 6: having a retention time RT of 45.385, an RSD of 0.6%, and a peak area of 434.784, which accounts for 1.474% of the total peak area; and

Peak 7: having a retention time RT of 54.011, an RSD of 0.8%, and a peak area of 7220.747, which accounts for 24.475% of the total peak area.

### Embodiment 6

Embodiment 6 of the present invention discloses fingerprint determination for an antidepressant traditional Chinese medicine composition. The adopted technical solution is as follows:

Raw materials: 3 g of Hypericum perforatum L., 12 g of Paeonia lactiflora Pall, 6 g of Gardenia jasminoides Ellis, 10 g of Albizia julibrissin Durazz. and 10 g of Moutan Cortex.

1. Preparation Method

Adding 10 times the amount of 60% ethanol to the Hypericum perforatum L., conducting heating reflux extraction twice for 1.5 h each time, merging the extracting solutions, concentrating, and drying under reduced pressure; adding 10 times the amount of 70% ethanol to the Paeonia lactiflora Pall, conducting heating reflux extraction twice for 1.5 h each time, merging the extracting solutions, concentrating; and drying under reduced pressure; adding 10 times the amount of 80% ethanol to the Gardenia jasminoides Ellis, conducting heating reflux extraction twice for 1.5 h each time, merging the extracting solutions, concentrating, and drying under reduced pressure; adding 10 times the amount of pure water to the Albizia julibrissin Durazz., conducting heating reflux extraction twice for 1.5 h each time, merging the extracting solutions, concentrating, and drying under reduced pressure; adding 10 times the amount of pure water to the Moutan Cortex, and obtaining an extract by steam distillation.

2. Main Instruments

Chromatographic system: Agilent Technologies 1260 Infinity II (G7111B 1260 Quat Pump, G7129A 1260 Vialsampler, G7116A 1260 MCT, G7117B 1290 DAD);

Ultrasonic cleaner: KQ-600DE, Kun Shan Ultrasonic Instruments Co., Ltd.;

Rotary evaporator: RE-52A, Shanghai Yarong Biochemistry Instrument Company

Circulating water vacuum pump: SHZ-D(III), Gongyi Yuhua Instrument Co., Ltd.

3. Reagents

Methanol: 20180606, purity≥99.9%, Sinopharm Chemical Reagent Co., Ltd.;

Acetonitrile: 20180731, purity≥99.9%, Sinopharm Chemical Reagent Co., Ltd.;

Phosphoric acid: purity≥85%, Tianjin Kermel Chemical Reagent Co., Ltd.;

Purified water: 20180927, A.S. Watson Group.

4. Chromatographic Conditions:

A chromatographic column adopts octadecylsilane chemically bonded silica as a filler; gradient elution is adopted; mobile phase A is acetonitrile; mobile phase B is phosphoric acid water; detection wavelengths are 230 nm, 238 nm, 256 nm, 274 nm, 300 nm; flow rate is 1 ml/min; column temperature is 30° C.; sample injection volume is 10 μL; gradient elution procedures are:

| Time, min Mobile Phase A %, | Acetonitrile Mobile Phase B %, | 0.1% Phosphoric Acid |
|---|---|---|
| 0 | 5 | 95 |
| 5 | 5 | 95 |
| 15 | 14 | 86 |
| 25 | 14 | 86 |
| 30 | 15 | 85 |
| 35 | 15 | 85 |
| 40 | 20 | 80 |
| 75 | 20 | 80 |
| 80 | 25 | 75 |
| 90 | 25 | 75 |
| 95 | 37 | 63 |
| 115 | 37 | 63 |
| 135 | 90 | 10. |

5. Determination

Sucking the sample solution of the Traditional Chinese medicine composition and injecting same in a liquid phase chromatograph, determining according to high performance liquid chromatography, and obtaining a fingerprint of the traditional Chinese medicine composition, wherein the fingerprint peaks include:

Peak 1: having a retention time of 9.153, an RSD of 0.08%, and a peak area of 1336.402, which accounts for 4.432% of the total peak area;

Peak 2: having a retention time of 11.695, an RSD of 0.04%, and a peak area of 521.328, which accounts for 1.729% of the total peak area;

Peak 3: having a retention time of 15.111, an RSD of 0.04%, and a peak area of 305.260, which accounts for 1.012% of the total peak area;

Peak 4: having a retention time of 16.559, an RSD of 0.04%, and a peak area of 437.930, which accounts for 1.452% of the total peak area;

Peak 5: having a retention time of 19.563, an RSD of 0.06%, and a peak area of 406.334, which accounts for 1.348% of the total peak area;

Peak 6: having a retention time of 20.383, an RSD of 0.04%, and a peak area of 1151.954, which accounts for 3.820% of the total peak area;

Peak 7: having a retention time of 32.511, an RSD of 0.04%, and a peak area of 820.504, which accounts for 2.721% of the total peak area;

Peak 8: having a retention time of 44.247, an RSD of 0.09%, and a peak area of 1617.883, which accounts for 5.365% of the total peak area;

Peak 9: having a retention time of 44.856, an RSD of 0.07%, and a peak area of 1299.504, which accounts for 4.309% of the total peak area;

Peak 10: having a retention time of 45.764, an RSD of 0.09%, and a peak area of 1299.504, which accounts for 4.433% of the total peak area;

Peak 11: having a retention time of 49.397, an RSD of 0.09%, and a peak area of 2051.489, which accounts for 6.803% of the total peak area;

Peak 12: having a retention time of 51.643, an RSD of 0.05%, and a peak area of 563.488, which accounts for 1.869% of the total peak area;

Peak 13: having a retention time of 53.324, an RSD of 0.03%, and a peak area of 895.859, which accounts for 2.971% of the total peak area;

Peak 14: having a retention time of 88.658, an RSD of 0.02%, and a peak area of 646.792, which accounts for 2.145% of the total peak area; and Peak 15: having a retention time of 96.463, an RSD of 0.04%, and a peak area of 9089.165, which accounts for 30.142% of the total peak area;

### Embodiment 7

Embodiment 7 of the present invention discloses verification of methodology. The adopted technical solution is as follows:

1. Preparation of Reference Solution

Taking an appropriate amount of hyperoside, and adding 50% methanol to prepare reference solutions with concentrations of 1 mg/ml, 0.5 mg/ml, 0.25 mg/ml, 0.125 mg/ml, 0.0625 mg/ml, 0.03125 mg/ml.

2. Preparation of Standard Curve and Determination of Linear Range

Taking an appropriate amount of hyperoside standard solution and filtering same via a 0.45 μm microporous membrane, according to the set chromatographic conditions, performing sample injection respectively with a sample injection volume of 10 μL, drawing a standard curve with the chromatographic peak area as the ordinate and the concentration as the abscissa, thus obtaining a linear regression equation and a linear range of hyperoside.

The regression equation of hyperoside is: Y=19800.1399X-148.0636, R²=0.99975; hyperoside has a good linear relationship with the peak area within the linear range.

3. Precision Experiment

Precisely sucking the above hyperoside standard solution, determining using HPLC, continuously performing sample injection five times with a sample injection volume of 10 μL each time, determining the RSD of the peak area thereof, and detecting the precision of the high-efficient liquid chromatograph thereof.

The RSD of the retention time of the hyperoside is 0.06%; and the RSD of the peak area is 1.07%.

4. Stability Experiment

Taking traditional Chinese medicine composition samples, determining using HPLC at 3 h, 6 h, 9 h, 12 h, 15 h according to the above chromatographic conditions respectively, the sample injection volume being 10 μL; integrating the peak area thereof, calculating the relative standard deviation of the integral value, and detecting the stability thereof.

The RSD of the retention time of the hyperoside is 0.04%; and the RSD of the peak area is 5.05%.

5. Reproducibility Experiment

Taking the same batch of samples, preparing backup test solutions according to the preparation method of the test solution, determining using HPLC according to the above chromatographic conditions, the sample injection volume being 10 μL; integrating the area thereof, calculating the relative standard deviation, and detecting the reproducibility thereof.

The RSD of the retention time of the hyperoside is 0.09%; and the RSD of the peak area is 3.05%.

6. Sample Added Recovery Experiment

Weighing 5 parts of sample of the traditional Chinese medicine composition with known content, adding a certain amount of hyperoside reference substances respectively, preparing 5 parts of sample according to the preparation method of the test solution, continuously performing sample injection on all samples with a sample injection volume of 10 μL each time, determining the content using the standard curve method, and calculating the average sample added recovery of reference substances.

The RSD of the content of the hyperoside is 0.12%; and the RSD of the peak area is 2.19%.

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

1. A fingerprint detection method for an antidepressant drug, wherein liquid chromatography is adopted with chromatographic conditions are as follows:
a chromatographic column adopts octadecylsilane chemically bonded silica as a filler;
mobile phase A is acetonitrile, mobile phase B is phosphoric acid aqueous solution, and gradient elution is adopted;
detection wavelength is 230-300 nm;
flow rate is 1 ml/min;
sample injection volume is 10 μL;
the number of theoretical plates is based on peaks of standard hyperoside, ≥3000;
preparation of a standard solution: taking hyperoside, accurately weighing same, and adding 50% methanol aqueous solution to prepare a solution with a concentration of 1 mg/ml;
gradient elution conditions are as follows:

| Time, min Mobile Phase A %, | Acetonitrile Mobile Phase B %, | 0.1% Phosphoric Acid |
|---|---|---|
| 0 | 5 | 95 |
| 5 | 5 | 95 |
| 15 | 14 | 86 |
| 25 | 14 | 86 |
| 30 | 15 | 85 |
| 35 | 15 | 85 |
| 40 | 20 | 80 |
| 75 | 20 | 80 |
| 80 | 25 | 75 |
| 90 | 25 | 75 |
| 95 | 37 | 63 |
| 115 | 37 | 63 |
| 135 | 90 | 10. |

2. The fingerprint detection method for an antidepressant drug according to claim 1, wherein the antidepressant drug is composed of the following parts by weight of raw materials: 3 parts of Hypericum perforatum L., 12 parts of Paeonia lactiflora Pall, 6 parts of Gardenia jasminoides Ellis, 10 parts of Moutan Cortex and 10 parts of Albizia julibrissin Durazz.

3. The fingerprint detection method for an antidepressant drug according to claim 1, wherein the fingerprint of the antidepressant drug is:
the fingerprint comprises 15 common peaks: the relative retention times of all peaks are:
(1) 9.153, (2) 11.695, (3) 15.111, (4) 16.559, (5) 19.563, (6) 20.383, (7) 32.511, (8) 44.247, (9S) 44.856, (10) 45.764, (11) 49.397, (12) 51.643, (13) 53.324, (14) 88.658, (15) 96.463.

4. The fingerprint detection method for an antidepressant drug according to claim 1, wherein in the method, the pH of the mobile phase A is 2.0-7.0.

5. The fingerprint detection method for an antidepressant drug according to claim 1, wherein for a reference solution used for determination by liquid chromatography in the method, a preparation method thereof includes: taking hyperoside and putting same into a volumetric flask, adding methanol aqueous solution, conducting ultrasonic treatment for dissolution, making constant volume, filtering, thus obtaining a filtrate as the reference solution.

6. The fingerprint detection method for an antidepressant drug according to claim 1, wherein for a test solution used for determination by liquid chromatography in the method, a preparation method thereof includes: taking dry powder of the antidepressant drug and putting same into a volumetric flask, adding methanol aqueous solution, conducting ultrasonic treatment for dissolution, making constant volume, filtering, thus obtaining a filtrate as the test solution.

7. A preparation method for the test solution according to claim 6, wherein the methanol aqueous solution is 40%-60% methanol solution.
